(19) United States
(12) Patent Application Publication
(10) Pub. No.: US 2011/0230755 A1
(43) Pub. Date: Sep. 22, 2011

(54) Single camera motion measurement and monitoring for magnetic resonance applications

(76) Inventors: Duncan MacFarlane, Dallas, TX (US); Chester R. Wildey, Euless, TX (US)

(21) Appl. No.: 12/932,733

(22) Filed: Mar. 4, 2011

Related U.S. Application Data
(60) Provisional application No. 61/310,703, filed on Mar. 4, 2010.

(51) Int. Cl. A61B 5/055

(52) U.S. Cl. 600/414

(57) ABSTRACT

An optically-based rigid-body 6-DOF motion tracking system optimized for prospective (real-time) motion correction in Magnetic Resonance Imaging (MRI) applications using a single camera with an on-board image processor, an IR illuminator and optical fiducial targets affixed to a patient. An angle extraction algorithm operated by the on-board image processor utilizes successive approximation to solve the 3-point pose problem for angles close to the origin to achieve convergence to sub-microradian levels. A motion alarm is enabled by a monitor and GUI application in communication with the motion tracking system. A motion correction is enabled for MR scan images taken while operating the motion tracking system wherein an MRI controller is in communication with the motion tracking system.

### CROSS-REFERENCE TO RELATED APPLICATIONS

This application claims priority to Provisional Patent Application No. 61/310,703 filed on Mar. 4, 2010.

### FIELD OF THE INVENTION

The present disclosure relates to measurement and monitoring of magnetic resonance imaging. In particular, the disclosure relates to an optical system for measurement of the position of a rigid body in space adapted for applications in the field of magnetic resonance imaging.

### BACKGROUND OF THE INVENTION

Computerized tomography (CT), magnetic resonance imaging (MRI), and positron emission tomography (PET), coupled with developments in computer-based image processing and modeling capabilities have led to significant improvements in the ability to visualize anatomical structures in human patients. This information has become invaluable in the diagnosis, treatment, and tracking of patients. The technology has been recently been expanded to be used in conjunction with real-time interventional procedures.

MRI is a known method of creating images (referred to as MR images) of the internal organs in living organisms. The primary purpose is demonstrating pathological or other physiological alterations of living tissues. MRI has also found many niche applications outside of the medical and biological fields such as rock permeability to hydrocarbons and certain non-destructive testing methods such as produce and timber quality characterization. Superb image contrast for soft tissues and high spatial resolution have established MRI as a prepared imaging technology. MRI is unique in that many tissue properties can be simultaneously observed.

The MRI process requires a highly accurate and stable target. This is a consequence of the process by which medical MRI functions. Medical MRI relies on the relaxation properties of excited hydrogen nuclei in water. When an object is placed in a powerful, uniform magnetic field, the spins of the atomic nuclei with non-zero spin numbers align in one of two opposite directions: parallel to the magnetic field or antiparallel.

The difference in the number of parallel and antiparallel nuclei is only about one in a million. However, due to the vast quantity of nuclei in a small volume, the nuclei sum to produce a detectable change in field strength. The magnetic dipole moment of the nuclei then moves in a gyrating fashion around the axial field. While the proportion is nearly equal, slightly more nuclei are oriented at the low energy angle. The frequency with which the dipole moments process is called the Larmor frequency. The tissue is then briefly exposed to pulses of electromagnetic energy (RF pulse) in a plane perpendicular to the magnetic field, causing some of the magnetically aligned hydrogen nuclei to assume a temporary non-aligned high-energy state.

In order to selectively image the different voxels (3-D pixels) of the material in question, three orthogonal magnetic gradients are applied, slice selection, phase encoding and frequency encoding. Usually, the three gradients are applied in the X, Y, and Z directions. Any small shift in the position of the patient with respect to these fixed gradient axes will alter the orientations and positions of the selected slices and result in poor imaging.

In order to create an MR image, spatial information must be recorded along with the received tissue relaxation information. For this reason, magnetic fields with an intensity gradient are applied in addition to the strong alignment field to allow encoding of the position of the nuclei. A field with the gradient increasing in each of the three dimensional planes is applied in sequence. This information is then subsequently subjected to a Fourier transformation by a computer that transforms the data into the desired image and yields detailed anatomical information results.

With conventional anatomic MR imaging, the presence of moving biological tissue is problematic. The tissue produces image artifacts, degrades the quality of the images, and complicates the interpretation of MR images. The typical appearance of such image artifacts takes the form of “blurring,” or a characteristic “motion ghost” in the phase encoding direction associated with incorrectly encoding the spatial frequencies of a moving object that is assumed to be static.

Through the process of MRI, anatomy can be defined in great detail, and several other biophysical and metabolic properties of tissue, including blood flow, blood volume, elasticity, oxygenation, permeability, molecular self-diffusion, anisotropy, and water exchange through cell membranes, can also be represented. Conventional anatomical MR imaging uses this spin-echo, gradient-echo, and inversion recovery sequencing. There are other methods of MR that are currently being used, including magnetic resonance spectroscopy (MRS), apparent diffusion coefficient (ADC) mapping, diffusion-weighted imaging (DWI) and its derivatives of diffusion tensor imaging and tractography, perfusion imaging, permeability imaging, MR angiography (MRA), and functional MRI (fMRI). As the techniques of MR become more precise, there is corresponding need for increased accuracy and the tracking of the patient during the MR procedure. See, E. FUKUSHIMA AND S. B. W. ROEDER, EXPERIMENTAL PULSE NMR (Addison-Wesley, Reading, Mass. 1981); T. C. FARRAR, AN INTRODUCTION TO PULSE NMR SPECTROSCOPY (Farragut Press, Chicago, 1987); R. C. JENNISON, FOURIER TRANSFORMS AND CONVOLUTIONS (Pergamon Press, NY 1961); E. O. BRIGHAM, THE FAST FOURIER TRANSFORM (Prentice-Hall, Englewood Cliffs, N.J. 1974); and A. CARRINGTON A. D. MCLACHLAN, INTRODUCTION TO MAGNETIC RESONANCE (Chapman and Hall, London 1967) which are each hereby incorporated by reference.

Functional MRI (fMRI) measures signal changes in the brain that are due to changing neural activity. An fMRI scan is completed at a low resolution but at a very rapid rate (typically once every 1-3 seconds). Increases in neural activity cause changes in the MR signal via a mechanism called the BOLD (blood oxygen level-dependent) effect. Increased neural activity causes a corresponding increased demand for oxygen, which is responded to by the vascular system, which increases the amount of oxygenated relative to deoxygenated hemoglobin. Because deoxygenated hemoglobin attenuates the MR signal, the vascular response leads to a signal increase that is related to the neural activity. The use of MRI to measure physiologic and metabolic properties of tissue non-invasively requires dynamic imaging to obtain time-series data.

The fMRI process relies on neurovascular coupling that results in transient increases in blood flow, oxygenation, and volume in the vicinity of neurons that are functionally activated above their baseline level. Signal changes due to the blood oxygenation-level-dependent (BOLD) effect are intrinsically weak (only several percent signal change from baseline at 4.0 T or less). In addition, as BOLD imaging is typically coupled with a repetitive behavioral task (e.g., passive sensory, cognitive, or sensorimotor task) to localize BOLD signals in the vicinity of neurons of interest, there is significant potential for fMRI to be confounded by the presence of small head motions. Specifically, such motion can introduce a signal intensity fluctuation in time due to intra-voxel movement of an interface between two different tissues with different MR signal intensities, or an interface between tissue and air. Random head motion decreases the statistical power with which brain activity can be inferred, whereas task-correlated motion cannot be easily separated from the fMRI signal due to neuronal activity, resulting in spurious and inaccurate images of brain activation. In addition, head motion can cause mis-registration between neuroanatomical MR and fMR images that are acquired in the same examination session. This latter point is important because the neuroanatomical MRI data serve as an underlay for fMRI color maps, and mis-registration results in mis-location of brain activity. An analogous problem exists for aligning anatomical and functional MR images performed on different days.

Lack of motion in current MRI examinations anatomic motion is very important. Most aspects of human motor system performance require the patient to execute a movement as part of the behavioral task that is imaged to visualize brain activity. Movements can be very simple (e.g., self-paced finger tapping) or more complex (e.g., visually-guided reaching). Such examinations require both that the desired movement is performed in a well-controlled or well-quantified fashion, and also that the movement does not induce task-correlated head motion that confounds the ability to observe brain activity using fMRI. Perhaps the most complicated scenario involves combining use of virtual reality (VR) technology with fMRI, to determine brain activity associated with VR tasks for assessment and rehabilitation of impaired brain function. Such applications are important because they provide the opportunity to visualize brain activity associated with tasks that generalize well to everyday behavior. For example, position tracking would be required to provide a visual representation of a virtual hand operated by a data glove in a virtual environment.

The problem of motion tracking within an fMRI environment has been well documented in published medical literature describing various aspects of motion detection and quantitation. See, Seto et al., NeuroImage, 14:284-297 (2001); Hajnal et al., Magn. Res. Med., 31: 283-291 (1994); Friston et al., Magn. Res. Med., 35:346-355 (1996); Bullmore et al., Human Brain Mapping, 7: 38-48 (1999); Bandettini et al., Magn. Res. Med., 30:161-173 (1993); Cox, Comp. Med. Res., 29:162-173 (1996); Cox et al., Magn. Res. Med., 42:1014-1018 (1999); Grootoonk et al., NeuroImage, 11:49-57 (2000); Freire et al., IEEE Trans. Med. Im., 21(5):470-484 (2002); Babak et al., Mag. Res. Lin., 19:959-963 (2001); Voldye et al., Magn. Res. Med., 41:964-972 (1999), which are each incorporated by reference.

As the clinical applications of MRI expand, there is a concurrent requirement for improved technology to visualize and determine the position and orientation of moving objects in the imaging field. Improvements in position tracking technology are required to advance the resolution and quality of the MRI, including the ability to image the anatomy of a patient, the imaging of tissue functions, the use of MRI data for other imaging tasks, and interventional applications.

For anatomical and functional MRI applications, as well as interventional MRI, there is the additional need to register data from other imaging systems to provide comprehensive and complementary anatomical and functional information about the tissue of interest. Data registration allows different images to be overlaid, or to'ensure that images acquired in different spatial formats (e.g., MRI, conventional x-ray imaging, ultrasonic imaging) can be used to view the same spatial location. While some algorithms exist for performing such registrations, computational cost would be significantly reduced by developing technology that enables data from multiple different imaging modalities to be inherently registered by measuring the patient's orientation in each image with respect to a common coordinate system.

By detecting, tracking, and correcting for changes in movement, data acquisition can be synchronized to a specific target. As a consequence, MR data acquisition is gated to a specific position of the target, and by implication, to a specific position of a specific target region.

U.S. Pat. No. 6,067,465 to Foo, et al. discloses a method for detecting and tracking the position of a reference structure in the body using a linear phase shift to minimize motion artifacts in magnetic resonance imaging. In one application, the system and method are used to determine the relative position of the diaphragm in the body in order to synchronize data acquisition to the same relative position with respect to the abdominal and thoracic organs to minimize respiratory motion artifacts. The time domain linear phase shift of the reference structure data is used to determine its spatial positional displacement as a function of the respiratory cycle. The signal from a two-dimensional rectangular or cylindrical column is first Fourier-transformed to the image domain, apodized or bandwidth-limited, converted to real, positive values by taking the magnitude of the profile, and then transformed back to the image domain. The relative displacement of a target edge in the image domain is determined from an auto-correlation of the resulting time domain information.

There is often a need in neuroimaging to examine changes in brain images over long periods of time, such as the waxing and waning of MS lesions, progressive atrophy in a patient with Alzheimer's disease, or the growth or remission of a brain tumor. In these cases, the ability to determine the position of anatomy as a function of time is extremely important to detect and quantify subtle changes. High-spatial resolution is a basic requirement of 3D brain imaging data for patients with neurological disease, and motion artifacts a consequence of movement during scanning pose a significant problem. If a patient does not stay completely still during MR neuroimaging the quality of the MR scan will be compromised.

Many of the advantages of MRI that make it a powerful clinical imaging tool are also valuable during interventional procedures. The lack of ionizing radiation and the oblique and multi-planar imaging capabilities are particularly useful during invasive procedures. The absence of beam-hardening artifacts from bone allows complex approaches to anatomic regions that may be difficult or impossible with other imaging techniques such as conventional CT. Perhaps the greatest advantage of MRI is the superior soft-tissue signal contrast available, which allows early and sensitive detection of tissue changes during interventional procedures.

MR is used for procedures such as “interventional radiology”, where images produced by an MRI scanner guide surgeons in a minimally invasive procedure. However, the non-magnetic environment required by the scanner, and the strong magnetic radio frequency and quasi-static fields generated by the scanner hardware require the use of specialized instruments. Exemplary of such endoscopic treatment devices are devices for endoscopic surgery, such as for laser surgery disclosed in U.S. Pat. No. 5,496,305 to Kittrell, et al, and biopsy devices and drug delivery systems, such as disclosed in U.S. Pat. No. 4,900,303 and U.S. Pat. No. 4,578,061 to Lemelson.

Prior art attempts at tracking motion using cross-correlation and other simple distance measurement techniques have not been highly effective where signal intensities vary either within images, between images, or both. U.S. Pat. No. 6,292,683 to Gupta et al. discloses a method and apparatus to track motion of anatomy or medical instruments between MR images. The invention includes acquiring a time series of MR images of a region of interest, where the region of interest contains the anatomy or structure that is prone to movement, and the MR images contain signal intensity variations. The invention includes identifying a local reference region in the region of interest of a reference image and acquired from the time series. The local reference region of the reference image is compared to that of the other MR images and a translational displacement is determined between the local reference region of the reference image and of another MR image. The translational displacement has signal intensity invariance and can accurately track anatomy motion or the movement of a medical instrument during an invasive procedure. The translational displacement can be used to align the images for automatic registration, such as in myocardial perfusion imaging, MRA, fMRI, or in any other procedure in which motion tracking is advantageous. One of the problems with this disclosure is that the application and implementation of this methodology has proven difficult.

U.S. Pat. No. 5,947,900 to Derbyshire, et al. and U.S. Pat. No. 6,559,641 to Thesen disclose different correction schemes. The first is a correlation coefficient is used to determine the translational displacement. The second converts images to a binary form by thresholding and cross-correlation to create a signal peak which is plotted as the translational displacement.

U.S. Pat. No. 6,516,213 to Nevo discloses a method to determine the location and orientation of an object, while the body is being scanned by magnetic resonance imaging (MRI). Nevo estimates the location and orientation of various devices (e.g., catheters, surgery instruments, biopsy needles) by measuring voltages induced by time-variable magnetic fields in a set of miniature coils, said time-variable magnetic fields being generated by the gradient coils of an MRI scanner during its normal imaging operation. However, the method disclosed by Nevo is not capable of position tracking when imaging gradients are inactive, nor is it capable of measurements outside the sensitive volume of the imaging gradients.

Other systems are known. For example, fast imaging is known to “freeze” motion within the fMRI acquisition time frame, in combination with use of head restraints to limit motion. It is still possible to achieve poor activation image quality if patients exhibit task-correlated motion. This problem is particularly manifest in specific patient populations (e.g. dementia, immediate post-acute phase of stroke). Furthermore, image-based coregistration algorithms suffer from methodological limitations. Consequently, the resulting co-registered images still can suffer from residual motion contamination that impairs the ability to interpret brain activity.

Another method of tracking the position of a patient in an MRI is disclosed in U.S. Patent Application 2005/0054910 to Tremblay, et al., published Mar. 10, 2005. In this approach, a reference tool is fixed to a stationary target as close as possible to the centre of the sensitive measuring volume of an MRI-compatible camera system. There are several drawbacks of this approach, including the requirement of a second “tracking” component that must be calibrated with a dummy object, the position ambiguity due to the configuration of this approach, and the inherent limitation of the resolution provided by this approach.

U.S. Pat. No. 6,879,160 to Jakab describes a system for combining electromagnetic position and orientation tracking with magnetic resonance scanner imaging. Jakab discloses a system where the location of a magnetic field sensor relative to a reference coordinate system of the magnetic resonance scanner is determined by a tracking device using a line segment model of a magnetic field source and the signal from a magnetic field sensor. However, resolutions provided by the Jakab invention are not precise.

There is consequently a need for improved patient movement tracking techniques in medical imaging. There is a need for improved patient movement tracking that can function in adverse environments including high strength magnetic and/or radio frequency fields without the tracking mechanism exerting its own RF pulse or magnetic field. There is a need for improved patient movement tracking techniques that can be performed in real time. In particular, but without limitation, there is a need for real time tracking of a patient's head position in a high field strength fMRI without disrupting the scanning by the fMRI.

### SUMMARY OF THE INVENTION

Disclosed is a monocular optical system and associated three point pose algorithm optimized for real-time motion tracking in MRI applications. The optical fiducial is lightweight and facilitates closely coupled patient motion measurements in six degrees of freedom (“6 DOF”). Angular and translational accuracies are in the range of 100 microradians (0.005 degrees) and 10-100 microns. Due to the nature of the target and the use of a single camera, the x, y translation resolutions are approximately an order of magnitude better than the z direction resolution. However, the z value specifications are still well within the requirements for motion tracking and correction in MRI applications, and if necessary, may be improved by adding an additional turning mirror in the system, thus eliminating the need for a perspective-based measurement for z translation.

For both inherent patient motions and those correlated with commanded tasks in fMRI, good temporal agreement is observed between motion parameters derived from the camera and those produced by the PACE algorithm for low frequency motions. The aliasing inherent in the MRI measurements is apparent for higher frequency motions, and the movements monitored by PACE diverge from those measured with the camera. These divergences in measured motion parameters promise improved image quality with the camera system implemented to provide real-time feedback to the scanner gradients for prospective motion correction.

In one aspect, the monocular optical system is used as a motion alarm incorporating a monitor and graphical user interface to provide for an audible and visible alarm of excessive patient motion based on a measured centroid motion of an optical fiducial target.

The placement and small footprint of the instrument accommodates a wide range of additional equipment that is demanded by the complex and sophisticated protocols that are required by MRI applications. The IR illumination system improves imaging time and accuracy without impacting the patient or imaging protocols.

### BRIEF DESCRIPTION OF THE DRAWINGS

FIG. 1 is a graphical diagram of the motion tracking instrument in the context of an fMRI protocol that includes a projector display for visual cues.

FIG. 2 is a first exemplary embodiment of a three-dimensional, rotation/translation centroid invariant optical fiducial target comprising three discs used to determine the six degrees of motional freedom.

FIG. 3A is a second exemplary embodiment of an optical fiducial target comprising one ring and three discs used to determine the six degrees of motional freedom.

FIGS. 3B, 3C, 3D and 3E are perspective drawings of system components. FIG. 3B shows the first exemplary embodiment optical and MRI readable fiducial target mounted onto eyeglasses. FIG. 3C shows a mirror and optical fiducial target mounted on a mock-patient in the MRI scanner tunnel. FIG. 3D shows a mirror and optical fiducial target mounted on a mock-patient in the MRI scanner tunnel.

FIG. 4 is a screen shot of a motion monitor GUI.

FIG. 5 is a flow chart for a first embodiment method of tracking motion of a patient during an MRI image scan.

FIG. 6 is a flow chart for a second embodiment method of tracking motion of a patient during an MRI image scan.

FIG. 7 is a flow chart for an on-camera processing function to determine positions of the 6-DOF for an optical fiducial target.

FIG. 8 is a flow chart for an angle extraction and estimation method.

FIG. 9 is a schematic diagram showing an excess of movement of a stalk-mounted disc under x, y and z axis rotation.

FIG. 10 includes three plots showing convergence for angles x, y and z, respectively, versus iteration from 1 to 20 cycles. Horizontal axis is loop iteration number; vertical axis is log 10 of error magnitude in radians.

FIG. 11 includes three contour plots showing convergence in performance of the angle extraction step for three cases of angle z and across a range of angles x and y. The contour units are log 10 norm of angle error in radians. The horizontal axis shows changes in angle x in radians. The vertical axis shows changes in angle y in radians. From top to bottom the plots show z angles of +0.31, 0 and −0.31 radians respectively. The dotted box represents an arc of approximately 0.62 radians (36 degrees) in x and y centered on the origin.

FIG. 12 is a measured error in translation using a single translation stage with no mechanical coupling to other stages.

FIG. 13 is a graph of a measured translation taken by a preferred embodiment of a motion tracking system during a test wherein an MR image scan was being performed while a subject was speaking.

FIG. 14 is a set of measured 6-DOF plots of differential motion vs. time for head nod movement of a subject in a 3T scanner.

FIG. 15 is a set of measured 6-DOF plots of differential motion vs. time for counting aloud for a subject in a 3T scanner.

### DETAILED DESCRIPTION

The exemplary embodiments disclosed find particular use in radiological scanning such as MRI scanning where image data is captured over a relatively long period of time (seconds to minutes) and motion artifacts cause image corruption. Although common in MRI scanning, the image distortion is especially problematic for patients with involuntary tremors, for young patients and for injured patients. Often these motion artifacts are not discovered until after the scan has completed, thus resulting in an immediate re-scan, or are later discovered by the radiologist necessitating rescheduling the patient for a rescan.

It is possible to determine rigid body positions of the 6-DOF motion using a single camera. Disclosed is a single camera motion measurement system capable of precise relative 6-DOF measurements in the scanner environment using no in-scanner calibration. A simple multiple disc optical fiducial target is used in combination with an IR illuminator and the single camera. A program for extracting rotational and translational motion implements a special case of the 3-point pose problem that has no ambiguity in measured motion parameters and that converges quickly in a successive approximation for a limited range of rotations.

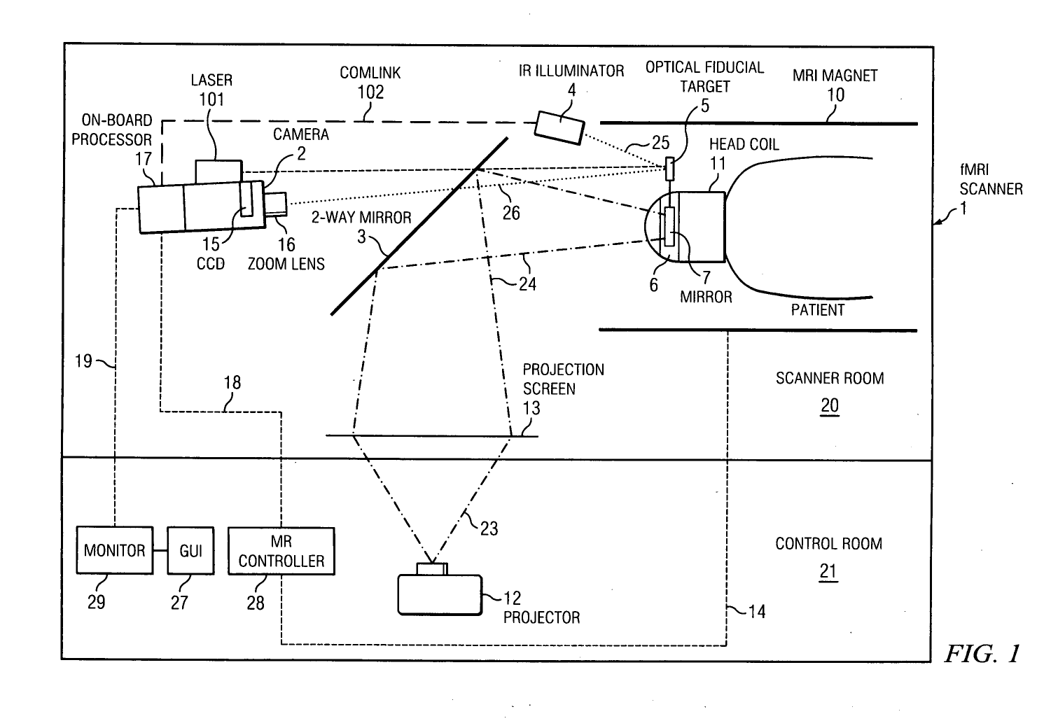

FIG. 1 shows fMRI scanner 1, comprising MRI magnet 10, head coil 11 and an associated projector display used to deliver visual stimuli for brain function experiments. The associated projector display comprises projector 12 projecting a stimulus image onto projection screen 13, two-way mirror 3 positioned to reflect the stimulus image into MRI magnet 10, and onto patient mirror 7 allowing a patient to view the stimulus image. Camera 2, fixed behind two-way mirror 3, comprises CCD detector 15, zoom lens 16 and onboard-processor 17. IR illuminator 4 is preferably an array of LEDs emitting LED light at approximately 800 nm directionally towards to optical fiducial target 5. The LED light is sufficiently intense to allow for short exposure times (typically 10 msec) and is invisible to the patient. Optical fiducial target 5 consists of non-reflective (absorptive) and retroreflective areas to ensure high contrast recorded optical images in camera 2.

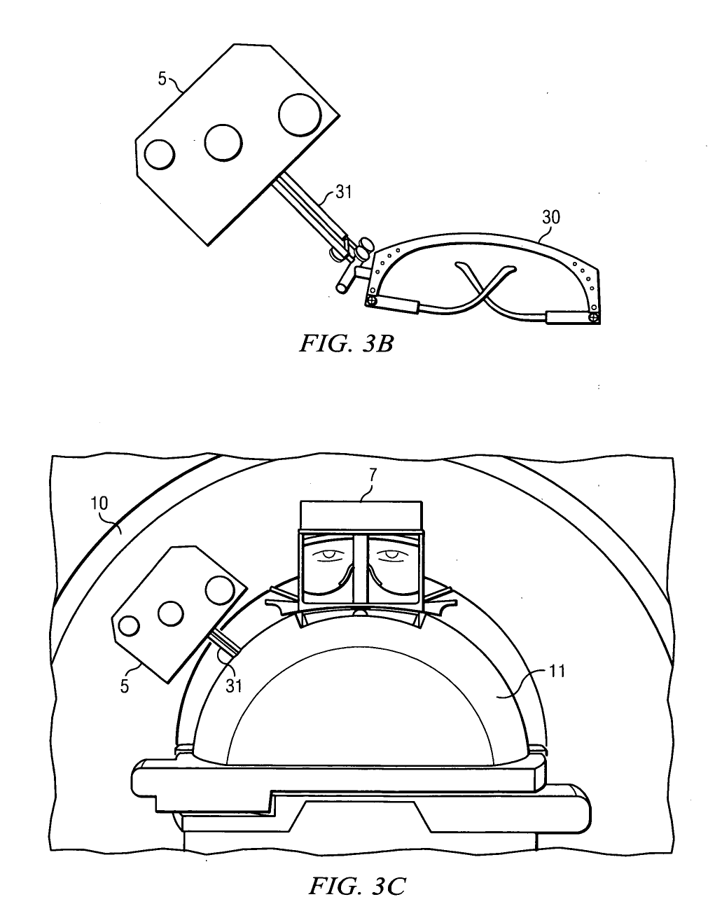

Projector 12 illuminates projector screen 13 along light path 23. Light path 24 is established between projector screen 13 and the patient eyes via two-way mirror 3 and patient mirror 7 mounted above head coil 11 and patient's head. Light path 25 is established between IR illuminator 4 and optical fiducial target 5. LED light from light path 25 reflects from optical fiducial target 5 along light path 26, through 2-way mirror to the CCD detector of camera 2. Optical fiducial target 5 is preferably mounted to the patient's head and protrudes from the side of head coil 11 through slot 6 preferably at an angle to keep the LED light and IR illuminator out of the field of view of the patient (see FIG. 3B).

Continuing with FIG. 1, fMRI scanner 1 is enclosed by an RF shield, usually in the form of a walled structure forming an enclosed MRI scanner room 20. Control room 21 for isolating electronic and metallic MR control components is adjacent to MRI scanner room 20. There are a set of communications links from MRI scanner room 20 to control room 21, penetrating the RF shield though a filtered penetration panel in the walled structure. Signals from fMRI components are sent via communications link 14 to controller 28.

In the exemplary embodiment, the fMRI scanner typically requires the display of photographic images to the patient to effect brain stimulation using a patient projection system such as the projector, screen and a patient mirror as described. In other embodiments involving radiological tests not requiring photographic brain stimulation, the two-way mirror is not utilized. The optical fiducial target can be positioned above the patient's head, if the projection system is not utilized, and to the side if the projection system is utilized.

Camera 2 is positioned nominally 2.5 m from the target. At this distance, the 3T magnetic field strength of MR magnet 10 falls off to the point where it does not substantially interfere with the electronics in the camera or the communication link to the control room. Also, at this distance, camera 2 does not interfere with the activities of nurses and technicians. Camera 2 is preferably constructed from metals such as copper, aluminum, brass, or a non-magnetic alloy as much as is possible to avoid interaction with the magnetic field of MR magnet. In the preferred embodiment camera 2 is Vision Components model VC4438.

Camera 2 is equipped with on-board processor 17 sufficient to enable calculation of 6-DOF data. On-board processing avoids transmission of image data to the controller or monitor over RF communication links in the scanner room. A communications link 18 is established between on-board processor 17 and controller 28. Another communication link 19 is established between on-board processor 17 and a motion monitor 29. An advantage of this architecture is the reduced communication bandwidth on communications links 18 and 19, which are typically serial communications links. In the preferred embodiment, suitable on-board processor 17 is the TI TMS 320 digital signal processor from Texas Instruments Corporation.

In an alternate embodiment, the CCD element is replaced by a CMOS image sensor.

In another alternate embodiment, camera 2 is equipped with a low-power laser pointer 101 (less than 5 mW) to aid the scanner operator in aligning the patient, the optical fiducial target, the IR illuminator and the camera.

In yet another embodiment, camera 2 is equipped with a shielded video cable connected to the motion monitor for sending a video signal to align the patient and optical fiducial target prior to an MR image scan. The video signal can be disabled by the scanner operator via the motion monitor to avoid interference with an MRI image scan.

IR illuminator is preferably controlled by the motion monitor, but can be controlled by on-board processor 17 though communications link 102 prior to and during an MR image scan in order to adjust contrast and backlighting levels for best performance. Alternatively, the IR illuminator can operate in a “constant on” fashion without control.

Motion monitor 29 is programmed to operate a graphical user interface, GUI 27, for alerting MRI personnel of unacceptable patient movement during an MR image scan. Motion monitor 29 is preferably a personal computer, which can be either a desktop computer or a laptop computer, configured to run the GUI and to interface with the MR controller.

Integration of the motion tracking system allows for images to be projected on a screen for visual fMRI stimuli. Two-way mirror 3, preferably a partially Al-coated glass substrate mirror (1.2 m×2.4 m), 85% reflecting and 15% transmitting to support high-quality optical imaging, is substituted for a standard high reflectivity mirror. Camera 2 thus remains invisible to the patient and obtains an unobstructed view down the magnet bore during the scan. The 6-DOF data is collected by camera 2 by imaging optical fiducial target 5 through two-way mirror 3.

Due to the invariance of their centroids under translation and rotation, a set of circular shaped optical objects offer good performance as the optical fiducial target. This is especially true for binary images, since as the image of the optical fiducial target moves across the CCD detector the time series of detected images progress through a series of representations with various pixels changing black/white states. The use of a set of circular shaped objects ensures that many pixels in the detected images do not change at once and that the reported position moves semi-smoothly as the target image translates across the CCD. Higher accuracy is found to be obtained with a set of at least three circular shaped objects covering a large area of camera 2 field of view. A higher zoom level, set by zoom lens 16, yields higher precision measurements, but at the cost of a lower range of movement. The position of camera 2 is preferably fixed so that zoom lens 16 can have a fixed focal length. Alternatively, zoom lens 16 has an adjustable focal length.

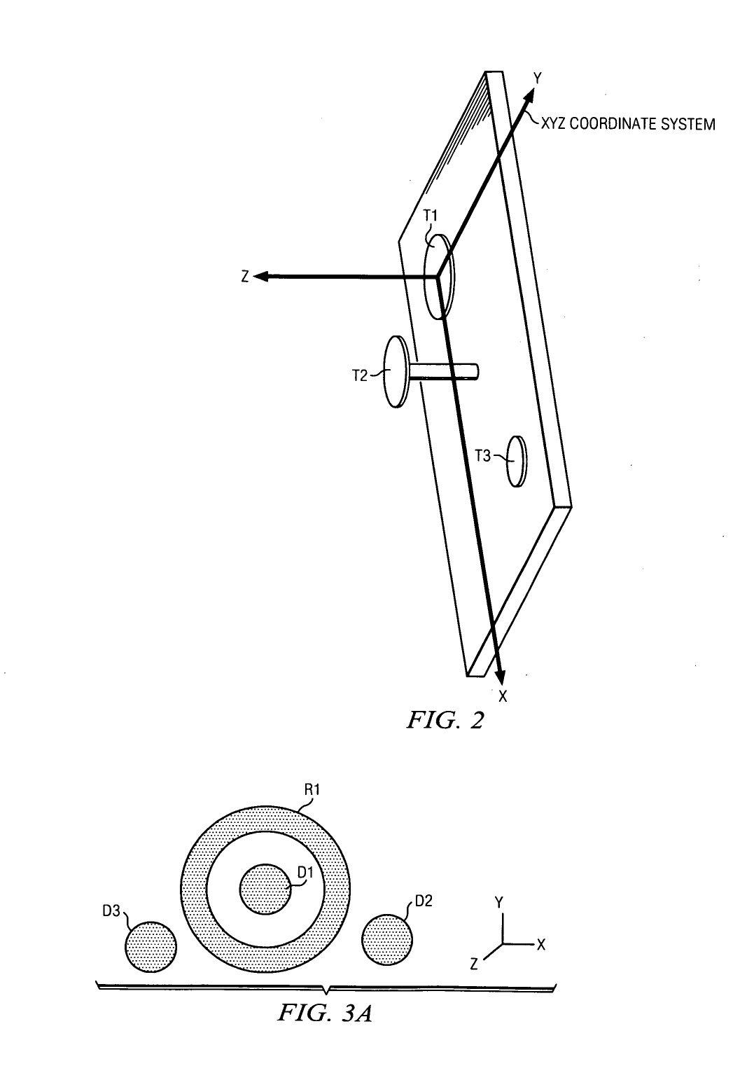

A preferred optical fiducial target is shown in FIG. 2. Optical fiducial target 5 comprises three circular discs, T1, T2 and T3 of high reflectivity or retro-reflectivity mounted on a dark background card for high contrast measurements. Only two of the three discs, T1 and T3, are coplanar in the xyz object coordinate system of the optical fiducial target, and the three circular discs must be visible without obscuration over the desired range of motion. In an alternate embodiment, the optical discs are replaced by highly reflective rings. In other alternate embodiments, more than three optical objects are used. In still another embodiment, the optical fiducial target utilizes water-filled components to enable increased coordination with the MRI machine.

FIG. 3A shows an alternate embodiment of an optical fiducial target pattern. The target pattern includes a ring R1, co-centric with a disc D1, protruding away from the xy plane of the xyz object coordinate system, and having a disc D2 and a disc D3 situated outside of ring R1. In a different embodiment, the disc D2 is not used.

The attachment of the optical fiducial target to the patient must meet a number of constraints including human factors, fMRI scanner constraints and metrology considerations. The target movements must faithfully correlate with the movement of a patient's head and brain for accurate image correction, which necessitates a relatively close and rigid attachment of the optical fiducial target to the patient's head. Since fMRI protocols may extend for one and even two hours, it is also essential that the attachment be comfortable to the patient. The attachment must also be quick and efficient to install due to scanner throughput considerations. A further constraint is that the optical fiducial target must be clearly visible to the camera through a compact and often cluttered scanner bore.

FIGS. 3B, 3C and 3D show three depictions of two exemplary optical fiducial targets in relation to fMRI system components. Beginning with FIGS. 3B and 3C, optical fiducial target 5 is mounted to eye glass frame 30. The eye glass frame contacts the patient at the bridge of the nose and behind the ears. These locations typically have skin that is thinner, less mobile, and more accessible than at other areas on the head. In some cases double sided tape or a similar adhesive is applied at these locations to further increase the stability of the optical fiducial target with respect to the patient. Eye glass frame 30 is preferably plastic or ceramic so as to not interfere with the MR scanner operation.

Referring to FIG. 3D, optical fiducial target 5 is attached via stalk 31 extending from the eye glass frame through a slot in head coil 11 situated in MRI magnet 10. The placement of optical fiducial target 5 to the side avoids a redesign of the patient mirror 7 ensuring that no impediment is introduced between the patient and the projector screen. Optical fiducial target 5 is preferably made of plastic or ceramic so as to not interfere with the MR scanner operation, however, water-filled components on the lower portion of the stalk enable the stalk as an MRI fiducial target which, when inside the head coil, can be imaged by the MRI scanner to allow transformation between the optical fiducial target and MRI scanner coordinate systems.

Referring to FIG. 3E, optical fiducial target 1010 is fixed to stalk 1005 which is in turn fixed to “bite-bar” 1000. In use, the patient secures the bite-bar in his teeth while imaging takes place.

A first embodiment of the motion measurement system includes a motion alarm. The motion measurement system detects patient movement in real time and provides immediate feedback to the scanner operator who may then take immediate corrective action such as termination of the scan early, settling the patient and performing a rescan. The immediate feedback is in realized in a first form as a motion alarm alerting scanner operator of patient movement in excess of a pre-defined adjustable threshold. The immediate feedback is realized in a second form as a trend graph of patient movement.

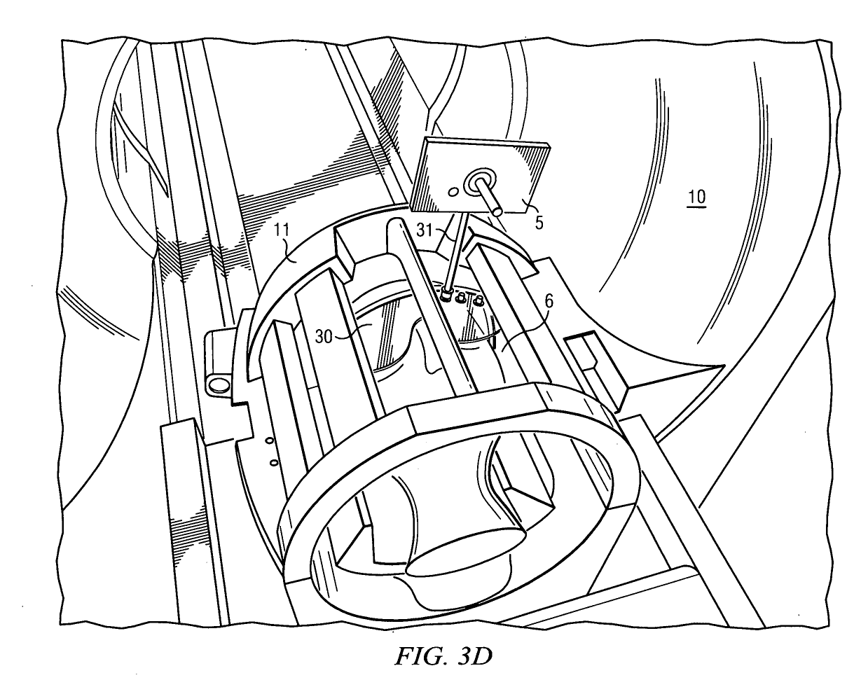

FIG. 4 shows a screen shot of the graphical display of GUI 27 configured for a motion alarm. GUI 27 comprises selector 33 for displaying a trend graph of patient motion, selector 32 for calibrating the thresholds for alarming, alarm indicator 34 in combination with an audio alarm to alert the MRI personnel, communications link indicator 35 for indicating quality of communications between camera 2 and motion monitor 29, and power indicator 36 indicating power to camera 2 and IR illuminator 4. The motion monitor preferably includes a touch screen display on which to operate the GUI.

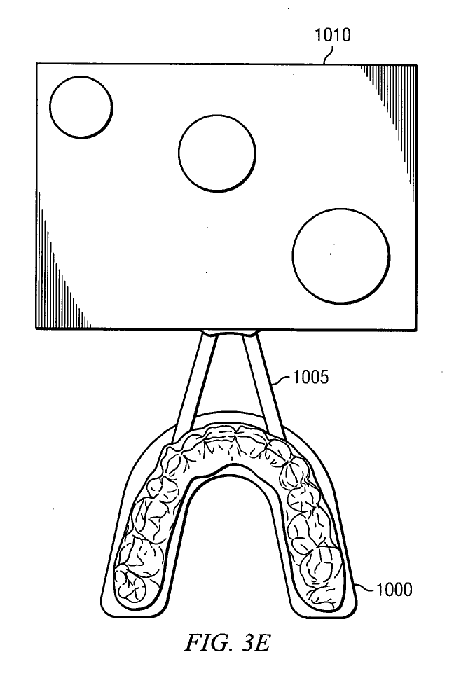

FIG. 5 shows a flow chart diagram of first embodiment method 60 of tracking motion of a patient during an MRI image scan. At step 58, a rotational threshold Rth is set to establish a maximum acceptable rotation of the optical fiducial target during an MR image scan. At step 59, a translational threshold Tth is set to establish a maximum acceptable translation of the optical fiducial target during an MR image scan. At step 61, a time series of images of the optical fiducial target is continuously collected from the camera during the MR image scan. At step 62, the on-board processor of the camera tracks the motion of the image centroid for the time series of images as centroid motion data. At step 63, the centroid motion data is sent to the monitor. If trend capability is enabled for the monitor at step 64, then at step 65, a graph of patient position is displayed on the GUI. If alarm capability is enabled for the monitor at step 66, then further checks are performed at steps 67 and 68. At step 67, if the centroid motion data indicates that the optical fiducial target is rotated more than Rth, then at step 69 an alarm is indicated on the GUI including a visible and an audible alarm. At step 68, if the centroid motion data indicates that the optical fiducial target is translated more than Tth, then at step 69 an alarm is indicated on the GUI including a visible and an audible alarm. Steps 61-66 are repeated during the MR image scan.

The trend capability preferably includes storage and trend calculations of 6-DOF data so that the graph of patient motion is allowed to extend into the past and preferably includes trend calculations using between 1 second and 1000 seconds of motion data.

A second embodiment of the motion measurement system is a motion tracking and correction system. The motion correction system detects patient movement in real time and provides real-time motion data to the MRI controller which takes immediate corrective action by adjusting the MRI images according to the motion data.

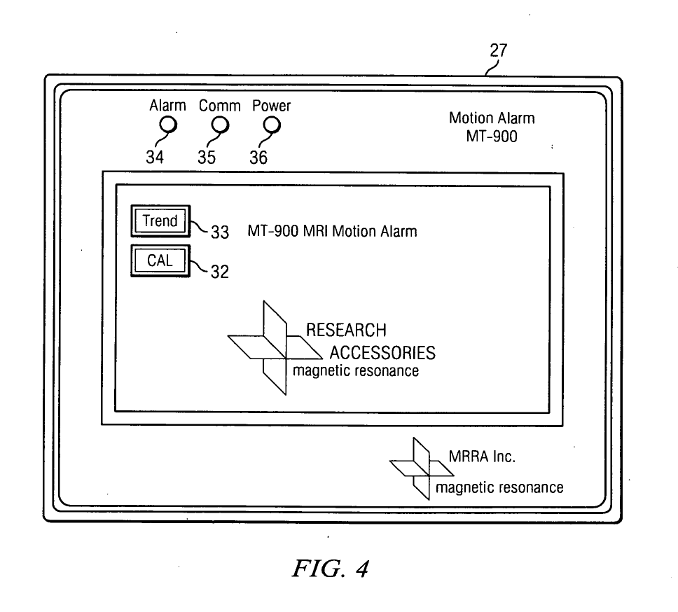

FIG. 6 shows a flow chart diagram of a second embodiment method 160 of tracking and correcting motion of a patient during an MRI image scan. At step 161, a time series of images of the optical fiducial target is continuously collected from the camera during the MR image scan. At step 162, the on-board processor of the camera tracks the motion of the image centroid for six degrees of freedom (6-DOF) for the time series of images. At step 163, the data for the 6-DOF is sent to the controller. If tracking capability is enabled in the controller at step 164, then at step 165, MRI images collected during the MR image scan are adjusted according to the patient motion characterized by the 6-DOF data. Steps 161-165 are repeated during the MR image scan.

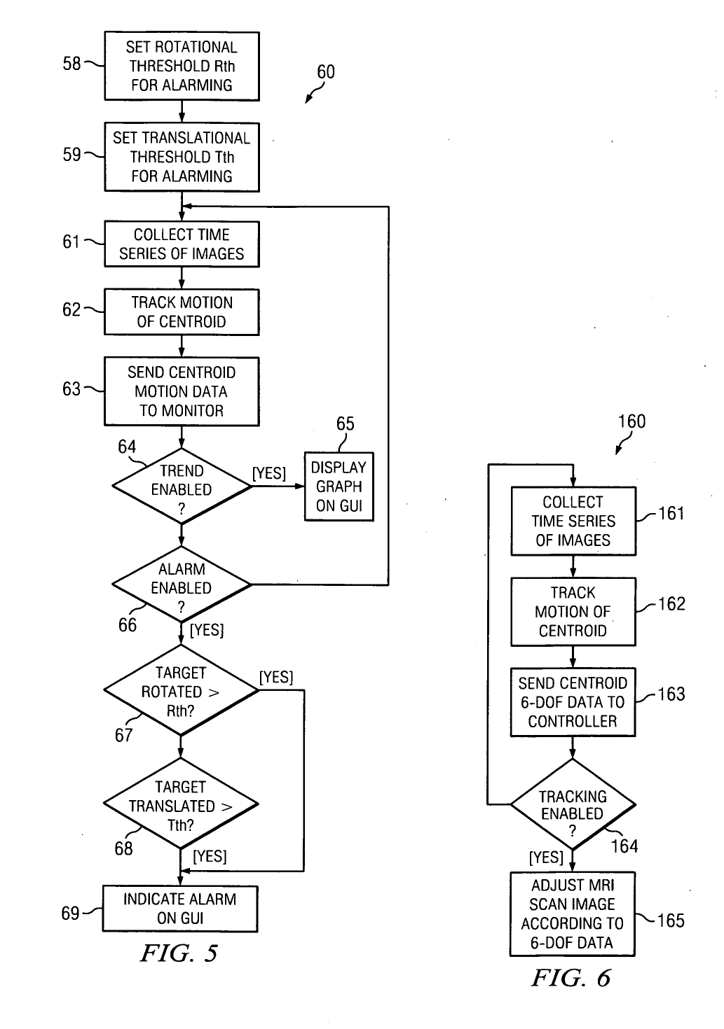

The on-camera processor is programmed to carry out a set of steps to continuously determine the three rotation angles and the three translation distances of the motion (6-DOF) of the optical fiducial target. In FIG. 7, a flow diagram of a preferred embodiment of on-camera processing function 40 to track centroid motion is shown.

At step 41, a preliminary signal processing of the raw CCD pixels is performed to threshold the image data and compress the image into binary pixels. The optical target identification step 42 then captures the image of the optical fiducial target including an image of the set of circular optical objects and an area filter discards any circular objects below a fixed area. At step 44, the centroids of the remaining circular optical objects are calculated in the order in which the circular objects are located and reported in step 42. The on-camera processing function searches for circular objects using a raster scan of data rows from upper left to lower right. Since the optical fiducial target is free to rotate with the patient's head, this introduces the possibility of inconsistent reporting order for the circular objects. To allow for inconsistent reporting orders, the physical areas of the circular objects (for example, the areas of T1, T2, T3 in FIG. 2) are made different. Step 44 sorts the centroid measurements by these areas.

Pincushion distortion of the lens, can cause errors in the precise determination of x and y coordinates of centroids and thus in the remaining four degrees of freedom. By modeling the effect of the pincushion as a calibrated parabaloid, these errors are numerically corrected in step 46.

In the descriptions of the remaining steps, an optical fiducial target having a set of three circular discs as in FIG. 2 are assumed in order to more clearly explain the steps. Other types of optical fiducial targets work in a similar manner. The centroid of each circular object is represented using three coordinates (xn, yn, zn) where n is an index to a particular circular object. The vector between a first and second circular objects is (x2-x1, y2-y1, z2-z1), and between the first and a third circular object is (x3-x1, y3-y1, z3-z1). “(x3-x1)” indicates a callipered measurement. “(x′3-x′1)” indicates a CCD measurement. As viewed by the CCD camera, these relative circular object measures are independent of translations in x and y (assuming proper camera and perspective calibration).

The distance X from the camera lens to the optical fiducial target is approximately known. A scale factor S is first calculated relating the number of pixels to a linear measure at each circular object. The scale factor allows calculation of the expected CCD-measured relative positions of the targets for a given set of rotation angles. At step 48 of perspective correction, the distance X and scale factor S are used to calculate the expected positions of the circular objects for zero rotation.

Equations 1, 2 and 3 define standard rotation matrices:

$$\begin{matrix}Rx = [ \begin{matrix}1 & 0 & 0 \\ 0 & cos ( {θ}_{x} ) & - sin ( {θ}_{x} ) \\ 0 & sin ( {θ}_{x} ) & cos ( {θ}_{x} )\end{matrix} ] & ( 1 ) \\ Ry = [ \begin{matrix}cos ( {θ}_{y} ) & 0 & sin ( {θ}_{y} ) \\ 0 & 1 & 0 \\ - sin ( {θ}_{y} ) & 0 & cos ( {θ}_{y} )\end{matrix} ] & ( 2 ) \\ Rz = [ \begin{matrix}cos ( {θ}_{z} ) & - sin ( {θ}_{z} ) & 0 \\ sin ( {θ}_{z} ) & cos ( {θ}_{z} ) & 0 \\ 0 & 0 & 1\end{matrix} ] . & ( 3 )\end{matrix}$$

Equation 4 relates the second and third circular object post-rotation positions relative to the first circular object in terms of their pre-rotation positions relative to the first circular object:

Rz Ry Rx (xₙ-x₁, yₙ-y₁, zₙ-z₁)^{T}=(x′ₙ-x′₁, z′ₙ-z′₁)^{T} (4)

where Rz, Ry, and Rx are the rotation operators, n is an object index, x, y, and z are the expected CCD measures of the circular object centroids at zero rotation for a given lens focal length and camera-to-fiducial target distance, and x′, y′, z′ are the circular object relative positions as measured directly from the CCD pixels.

The rotation angles θx, θy and θz satisfy the relation:

Rz Ry Rx (xₙ-x₁, yₙ-y₁, zₙ-z₁)^{T}−(x′ₙ-x′₁, y′ₙ-y′₁, z′ₙ-z′₁)^{T}=0 (5)

where θx is the rotation angle about the x-axis, θy is the rotation angle about the y-axis and θz is the rotation angle about the z-axis.

For n=2 and n=3 (second and third circular objects) this yields a coupled system of six equations, shown expanded below as Equations (6) through (11):

{(x₂-x₁)cos(θy)−[−(y₂-y₁)sin(θx)+(z₂-z₁)cos(θx)] sin(θy)} cos(θz)+[(y₂-y₁)cos(θx)+(z₂-z₁)sin(θx)] sin(θz)−(x′₂-x′₁)=0 (6)

−{(x₂-x₁)cos(θy)−[−(y₂-y₁)sin(θx)+(z₂-z₁)cos(θx)] sin(θy)} sin(θz)+[(y₂-y₁)cos(θx)+(z₂-z₁)sin(θx)] cos(θz)−(y′₂-y′₁)=0 (7)

(x₂-x₁)sin(θy)+[−(y₂-y₁)sin(θx)+(z₂-z₁)cos(θx)] cos(θy)−(z′₂-z′₁)=0 (8)

{(x₃-x₁)cos(θy)−[−(y₃-y₁)sin(θx)+(z₃-z₁)cos(θx)] sin(θy)} cos(θz)+[(y₃-y₁)cos(θx)+(z₃-z₁)sin(θx)] sin(θz)−(x′₃-x′₁)=0 (9)

−{(x₃-x₁)cos(θy)−[−(y₃-y₁)sin(θx)+(z₃-z₁)cos(θx)] sin(θy)} sin(θz)+[(y₃-y₁)cos(θx)+(z₃-z₁)sin(θx)] cos(θz)−(y′₃-y′₁)=0 (10)

(x₃-x₁)sin(θy)+[−(y₃-y₁)sin(θx)+(z₃-z₁)cos(θx)] cos(θy)−(z′₃-z′₁)=0 (11)

This system may not be solved directly since the equations contain CCD-measured z coordinates. A single CCD camera can only directly locate the x and y coordinates of the circular objects with the required fidelity. A rough estimate of z is possible using perspective and known fiducial target geometries; however, this estimate is not suitable for high definition 6-DOF measurements. The coupling of Equations (6)-(11), as well as the lack of a CCD z measurement, requires an iterative successive approximation to solve. At step 50, the rotation angles are extracted by estimating θx, θy and θz given a set of x′ and y′ CCD measurements of the circular object centroids by successive approximation in an iterative loop.

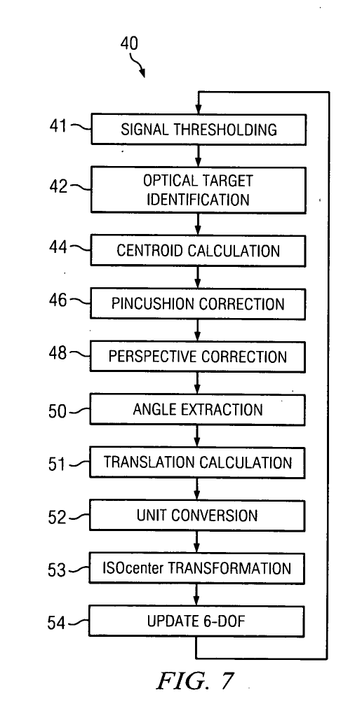

FIG. 8 shows the substeps of step 50 to extract rotational angles. At step 70, θx (0) and θy (0) are estimated, assuming zero for the other two angles and using standard numerical methods, such as Newton's method in combination with Eq. 7 and Eq. 6, respectively, to find zeros of the resulting equation. For example, to estimate θx (0), θy and θz are set to zero in Eq. 7. x1, x2, y1, y2, z1, and z2 are substituted in Eq.7 as the known positions of the objects included in the optical fiducial target. The camera measures y′2 and y′1 which is substituted in Eq. 7. A non-linear equation results with one unknown θx which is solved for iteratively by Newton's method. Note the pixel units are preferably used in step 50 for the linear coordinates, although a unit conversion to metric units could be performed as a part of step 50.

At step 72, θx(0) and θy(0) are compared. If θx(0) is found to be larger, θx (1) is set to θx (0) and step 76 is performed to estimate θy(1), substituting θx (1) for θx and θz=θz (1)=0 in Eq. 6 and solving iteratively for θy(1) using Newton's method. At step 78, θz(1) is estimated using the previously estimated values for θx(1) and θy(1) by solving Eq. 10 iteratively for θz (1) using Newton's method. Steps 74, 76 and 78 are repeatedly performed in a loop using successive angle estimates to recalculate θx(k), θy(k) and θz(k) for k iterations. The loop is checked for termination at step 80 wherein steeps 74, 76 and 78 are repeated until a set number of iterations have been reached or until the change in the estimates falls below a predetermined value. The change in estimates can be characterized for example by the sum |θx(k)-θx(k−1)|+|θx(k)-θx(k−1)|+|θz(k)-θz(k−1)| for the kth iteration. If the angle extraction step is terminated at step 80, the rotation angles θx, θy, and θz are reported as θx(k), θy(k) and θz(k). Note that at step 74, the angle θx(k) is estimated using θy(k−1) and θz(k−1).

If θy(0) is found to be larger than θx (0) at step 72, then θy (1) is set to θy (0) and step 86 is performed to estimate θx(1), substituting θy (1) for θy and θz=θz (1)=0 in Eq. 7 and solving iteratively for θx(1) using Newton's method. Next, at step 88, θz(1) is estimated using the previously estimated values for θx(1) and θy(1) by solving Eq. 9 iteratively for θz (1) using Newton's method. Steps 84, 86 and 88 are repeatedly performed in a loop using successive angle estimates to recalculate θy(k), θx(k) and θz(k) for k iterations. The loop is checked for termination at step 90 wherein steps 84, 86 and 88 are repeated until a set number of iterations have been reached or until the change in the estimates falls below a predetermined value. The change in estimates can be characterized for example by the sum |θx(k)-θx(k−1)|+|θx(k)−θx(k−1)|+|θz(k)-θz(k−1)| for the kth iteration. If the angle extraction step is terminated at step 90, the rotation angles θx, θy, and θz are reported as θx(k), θy(k) and θz(k). Note that at step 84, the angle θy(k) is estimated using θx(k−1) and θz(k−1).

The rotational angle extraction process of step 50 is preferably limited to 20 iterations with 10 iterations of Newton's method used for each angle estimation. For the preferred on-board camera processor, step 50 typically completes in less than 1 millisecond of execution time. The process terminates at step 91.

For an iterative method to converge to the correct solution, it is desirable that the error decrease with each iteration. For this reason the target geometry is selected such that the quantity (x′₂-x′₁) chiefly responds to θy (with lesser influence by θx and θz) and that (y′₂-y′₁) is mostly a measure of θx (with lesser influence by θy and θz).

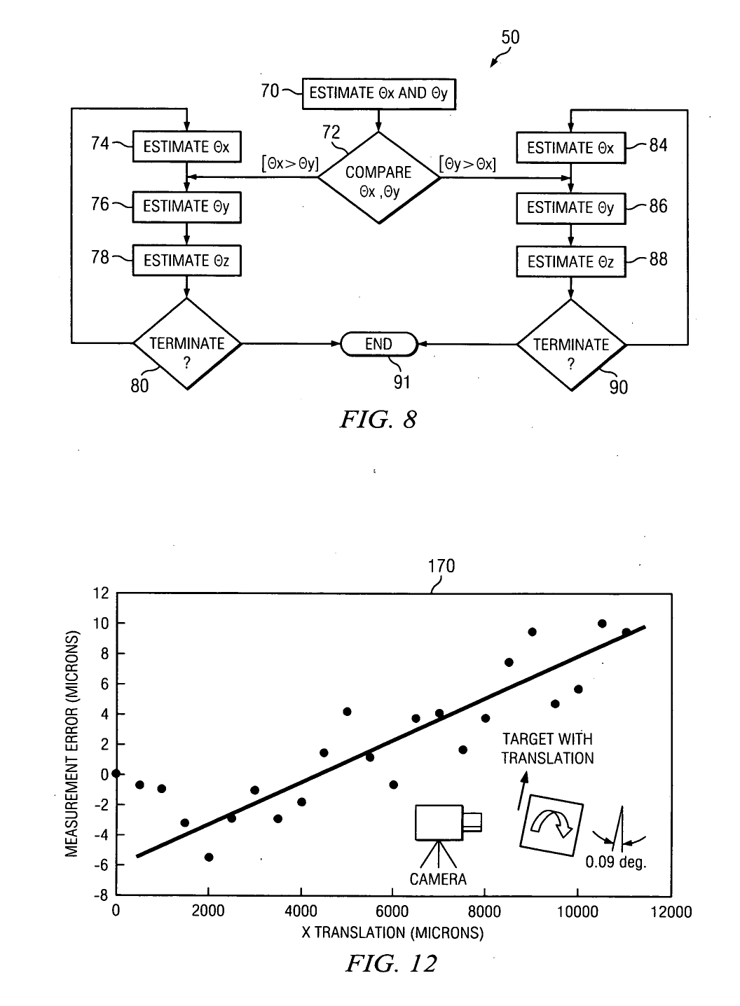

FIG. 9 shows an optical fiducial target under various rotations. At position 91, the fiducial target is seen with 0° rotation in the x and y directions. At position 92, the fiducial target is seen to be rotated approximately 10° about the y axis. At position 93, the fiducial target is rotated approximately 30° about the y axis. At position 94, the fiducial target is rotated approximately 15° about the x axis. Similarly, at position 95, the fiducial target is shown rotated approximately 30° about the x axis. At position 96, the fiducial target is rotated about the z axis by approximately 30° resulting in a rotational mix between Theta (y) and Theta (x). It can be seen that for small angles, (x′₂-x′₁) mostly responds to θy and that (y′₂-y′₁) mostly responds to θx while θz causes a mixing of this relation. Error reduction in each iteration is achieved for this particular target geometry as long as θz is not too large. For equipartition of the angles, the desired condition is met for some small range of θx, θy and θz around the origin. The convergence region of angle extraction step 50 as determined using a numerical simulation. In general, for equipartitioned rotation angles, the movement must lie within a half-radian (ca. 30 degree) arc centered on the origin. Since patient motion is typically constrained to less than this value, the convergence range is sufficient for most radiological measurements.

Returning now to FIG. 7, at step 51, the x and y translations are recovered directly as the x′1 and y′1 image coordinates. The z translation is recovered using a perspective relation incorporating the known geometry of the optical fiducial target. For the fiducial target shown in FIG. 2, the fiducial T1 to T3 distance D is preferably used to extract the z translation by first applying the previously determined rotation angles to rotate the CCD image coordinates to θx, θy and θz=0, then calculating the measured distance D′ between T1 and T3, and comparing the measured distance D′ to fiducial distance D in a simple geometrical relation relating the approximate target to lens distance L, the fiducial distance D, the measured distance D′ and the image magnification m:

z′1=(D−mD′)L/D (12)

At step 52, all of the 6-DOF measurements are scaled from units of pixels to millimeters in target dimensions, using the target range and lens focal length. Step 52 completes the measurement of 6-DOF in the optical fiducial coordinate system.

In another embodiment system, the stalk of the optical fiduciary target incorporates MRI-readable fiducial marks at known locations. At step 53, the MRI scanner locates the MRI-readable fiducial marks during a calibration to determine the spatial relation between MRI-readable fiducial marks and the optical fiducial target. With the known and fixed spatial relation between the MRI-readmarks and the optical fiducial target, transformation of the 6-DOF measurements from the optical fiducial coordinate system to the MR scanner isocenter coordinate system is accomplished by the on-board image processor using a set of homogenous transforms.

At step 54, the 6-DOF are reported to the MR scanner controller in MR scanner isocenter coordinates (see step 163 of second embodiment method 160 in FIG. 6). Additionally or alternatively, at step 54, the 6-DOF is reported to the monitor (see in step 63 of first embodiment method 60, FIG. 5). On-camera processing function 40 is repeated for a subsequent set of images at Signal Thresholding step 41.

An operational test of the on-camera processing function was performed, wherein a target was rotated by a known amount and the rotation measured using a simulated CCD camera. The angle extraction step was run on the resulting data and the recovered angles were compared to the known rotations. For small angles the algorithm converges rapidly to the correct angle, with convergence to sub-microradian (sub milli-degree) error levels achieved within 20 cycles.

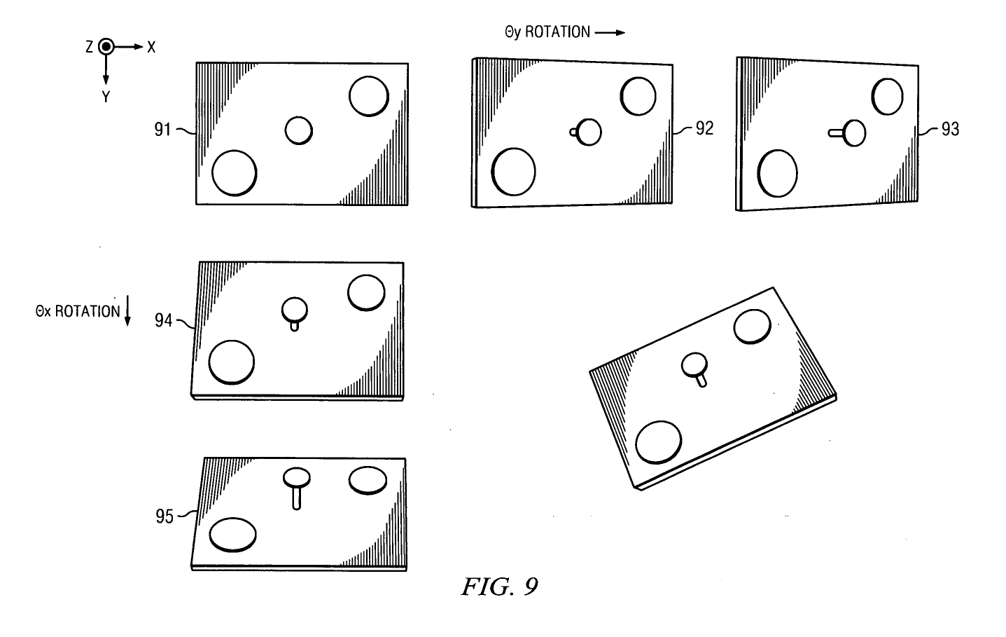

FIG. 10 shows the rate of convergence for θx in graph 110, for θy in graph 120, and for θz in graph 130, where θx, θy and θz=0.27 radians (15 degrees) with an error of 10E-16 radians in 20 iterations. The zero-rotation target coordinates for these simulations were T1=[0,0,0], T2=[10,10,20] and T3=[20,20,0] measured in pixels.

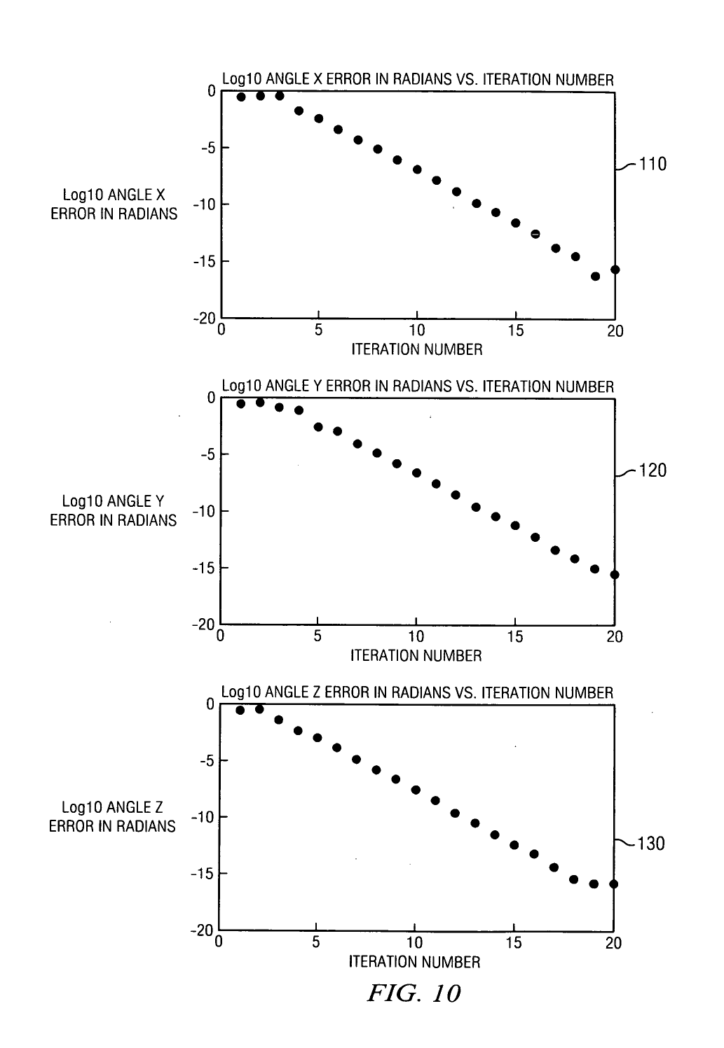

FIG. 11 shows contour plots of convergence errors for θx and θy rotations continuously varied over +/−1 radian (a 115 degree arc) for θz angles of −0.31 (graph 140), 0.0 (graph 145) and 0.31 radians (graph 150), corresponding to −18, 0, and 18 degrees, respectively. The contour lines are log 10 scale of convergence error in radians and illustrate the complex range of convergence, centered on the origin, of the algorithm. Larger ranges of convergence of θx and θy are possible if θz is constrained to a smaller range. Other target geometries are possible and will yield different performances. Central to system performance is the angle extraction algorithm. FIG. 11 shows the convergence of the algorithm over a range of approximately 2 radians (115 degrees). For accurate correction of MRI images the system measure angles with a maximum error of 1 milliradian (0.06 degrees). Allowing the angle extraction algorithm 10% of this error budget, the range of acceptable convergence as shown in FIG. 11 lies along the (−4) contour which corresponds to an error of 100 microradians (0.006 degrees).

In order to test the accuracy of pose determination and proper operation of the system a series of laboratory and scanner tests were performed. These included bench tests of calibrated motion and direct measurement of subject motion in the MRI scanner in both quiescent and task-based scenarios. Subject head motions measured by the optical camera were compared to MRI image-based motion parameters while bench tests were compared with callipered measurements.

First a static bench target was measured to establish the intrinsic noise of the measurement system. A stationary optical fiducial target was mounted to a pneumatically isolated floating optical table. Assuming complete vibration isolation by the table, this test measured the apparent motion caused by finite convergence of the algorithm combined with other hardware and software limitations, and benchmarks the best attainable precision of the instrument. The data for the static tracking experiment is shown in Table 1, column 1. The rms errors for angle measures were each <75 microradians (0.005 degrees) and for the x and y linear measures were less than 2 microns. The rms error for the z linear measure was 15 microns. The lower performance of the z linear measure is mainly due to the monocular measurement setup but is still much better than is required for radiological motion correction. For these tests, as for measurements in the scanner, the target was mounted 2.5 m from the CCD and a 75 mm focal length lens was used.

Two additional bench tests were made to determine linearity and accuracy of the motion tracking system for a moving target. For these tests, the fiducial was mounted on precision, calibrated motion-control stages and the motion measured by the camera was compared with the directly measured calibrated motion. The error between the two is a combination of impairments in the optical and the mechanical measurements.

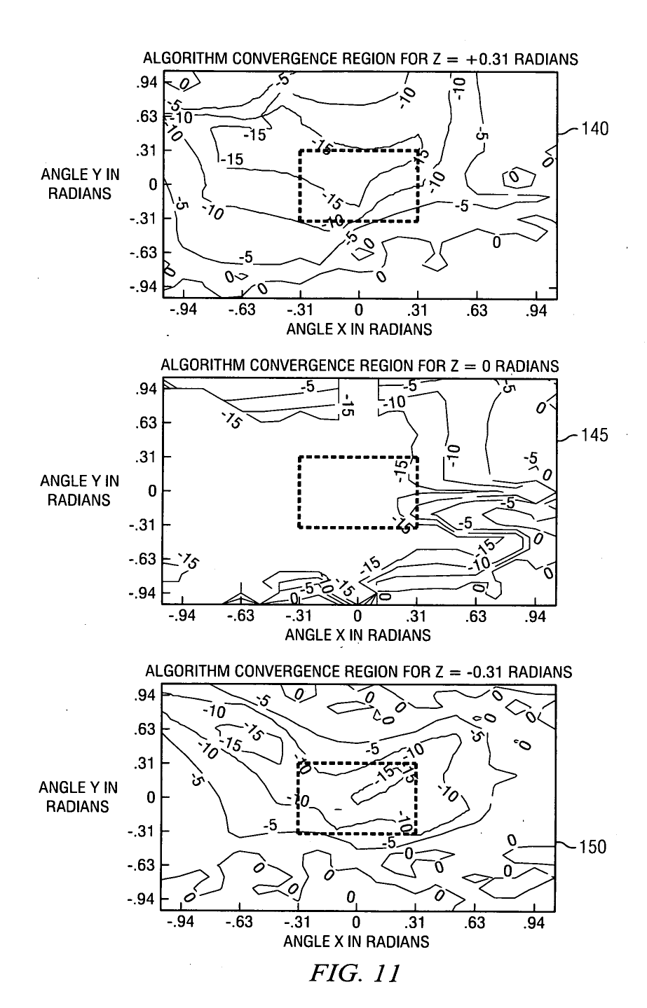

In FIG. 12, a plot of measurement error vs. x translation over 12 mm of travel for a single mechanical translation stage is shown in graph 170. According to graph 170, the error in this test accumulates linearly, reaching approximately 10 microns over 1 cm of travel. This is an absolute measurement with no linearization or calibration, including error due to the mechanics of the translation stage. One possible attribution of this error would be a misalignment between the camera coordinate frame and the translation stage frame. A misalignment of the translation stage of 1.6 milliradians (0.06 degrees) in the x-y plane is consistent with the previous, lower error found for measurement of a static target. Even if all of the error is attributed to the camera system, the performance is still much better than is required for motion correction. RMS of residual error from linear fit is 1.8 um. The error trend is consistent with foreshortening due to the 1.6 milliradian stage x-y misalignment (0.090 deg).

Since a system or algorithm error could manifest as a coupling between the various motions, a compound test jig comprising both translation and rotation stages was constructed. Testing was conducted using multiple successive rotations and translations. Over 1 cm translations the divergence from a linear fit, for x, y and z translations was 6.0, 4.0 and 108 microns rms, and over 100 milliradian (5 degree) rotations for θx, θy, and θz, the errors were 277, 170 and 232 microradians rms (0.016, 0.0097, and 0.013 degrees) respectively (Table 1, column 2). Since this is a multiply coupled mechanical system, these errors represent the camera measurement system error plus any errors due to the mechanics plus errors due to mechanical misalignment. This is a worst-case measure of the limits of system performance, and confirms the system is suitable for use in most MRI applications.

Obtaining calibrated motion in the MRI scanner environment is problematic due to the presence of strong magnetic fields. For example, a typical optical-bench micrometer constructed of steel would become a dangerous projectile when brought close to the scanner bore. For this reason a target was attached to the head coil of the scanner and the motion of the static target was observed, similar to the static bench test. The θx, θy, and θz, motions were less than 300 microradians rms (0.017 degrees); translational motion errors measured in x and y were less than 10 microns rms and for z were less than 100 microns rms (Table 1, column 3). This represents camera system errors plus any vibration induced motion caused by the scanner, which are within acceptable bounds.

TABLE 1

| | Static Bench | 6 Axis Mech. | Static Scanner |
|---|---|---|---|
| | RMS Error | RMS Error | RMS Error |
| X (microns) | 1.7 | 6.0 | 8.3 |
| Y (microns) | 1.8 | 4.0 | 5.1 |
| Z (microns) | 15 | 108 | 95 |
| θx (microradians) | 71 | 277 | 225 |
| θy (microradians) | 64 | 170 | 293 |
| θz (microradians) | 36 | 232 | 197 |

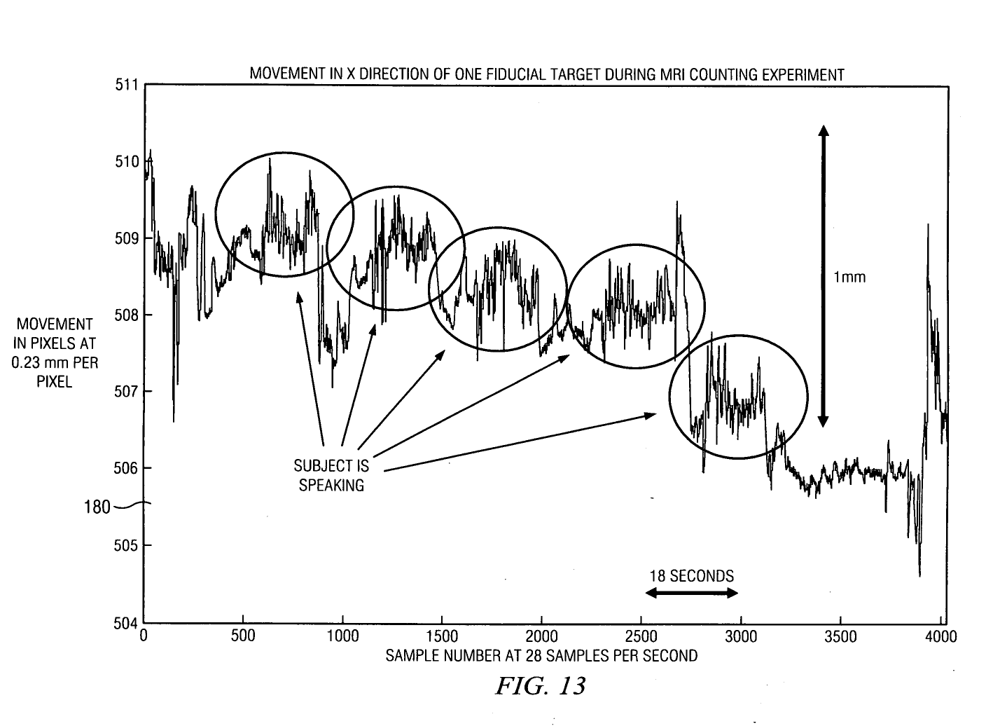

In another series of tests, the motion measurement system was operated during an MRI image scan while a patient was speaking aloud. In FIG. 13, graph 180 of centroid positions is shown where the x-direction of the xyz coordinate system of the optical fiducial target is sampled over time, the x-direction characterized by a number of image pixels for a range of time series samples taken within about 150 seconds. The speaking activity and motion of the subject is traceable by a change in image centroid position of about 5 pixels corresponding to about 1 millimeter in the xyz coordinate system of the optical fiducial target.

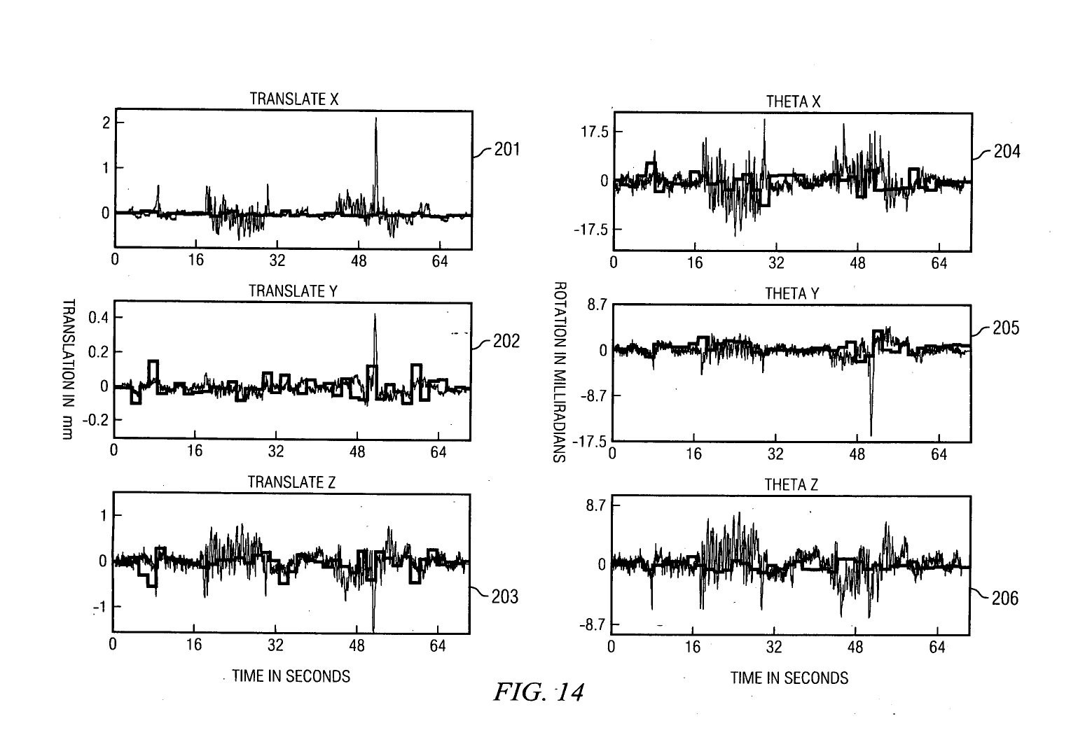

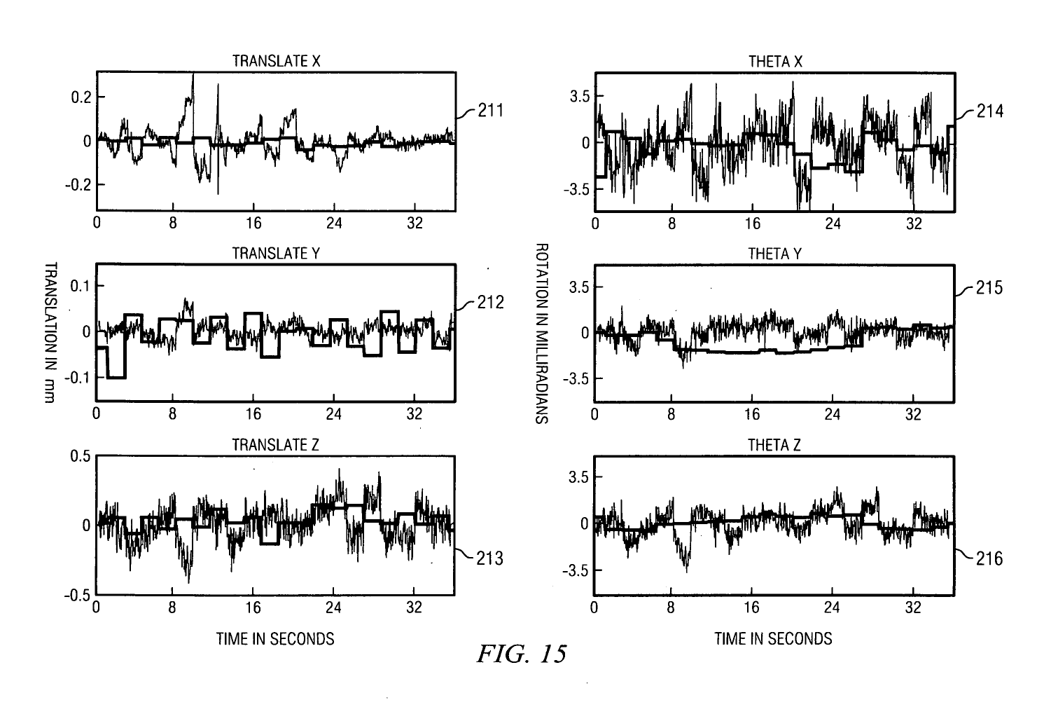

FIGS. 14 and 15 show motion responses for four subjects and twenty fMRI runs with durations of approximately 3 min 20 s, measured in experiments consisting of commanded patient head nodding (FIG. 14) and counting aloud (FIG. 15). Camera tracking measurements were compared to image-based motion measurements calculated using the PACE algorithm on a Siemens Magnetom 3 Tesla MRI scanner. The scanner-derived motion measurement was calculated every 2 seconds.

In FIG. 14, a time series plot measured by the camera is superimposed on the differential motion plot calculated from the PACE algorithm of the scanner. Head nodding occurred between approximately 18 and 30 seconds and between 43 and 56 seconds. These data are shown in graphs 201, 202, 203, 204, 205 and 206 of FIG. 14 for x, y, z, θx, θy and θz, respectively. In general, the camera tracking method and the scanner PACE algorithm method are in agreement, although the significantly better temporal resolution of the optical camera method provides a more accurate reading of faster motions.

In FIG. 15, a time series plot measured by the camera is superimposed on the differential motion calculated from the PACE algorithm of the scanner. Counting aloud occurred between approximately 7 and 20 seconds. These data are shown in graphs 211, 212, 213, 214, 215 and 216 of FIG. 15 for x, y, z, θx, θy and θz, respectively. In general, the camera tracking method and the scanner PACE algorithm method are in agreement, although the significantly better temporal resolution of the optical camera method provides a more accurate reading of faster motions.

Table 1 shows a comparison of system performance for static bench measurements (column 1), six axis dynamic bench test with calibrated motions (column 2) and static measure of scanner structure (column 3). Increased errors for the six axis tests are attributed to the more complex test setup causing mechanical error stack-up. Column 1 and 2 measurements were taken on a pneumatically vibration isolated optical bench. Column 3 data were taken with the scanner in idle mode and the target fixed to the scanner head coil. Errors for the movement test (column 2) are in relation to a best linear fit. Since image correction requires only differential position measurement, actual errors may be expected to be less than these by a factor of the square-root of two.

1. A method for correction of motion of an imaging target for a magnetic resonance scanner image where a magnetic resonance scanner includes a controller, the method comprising the steps of:
providing a fiducial target comprising a set of optical objects;
providing a source of infrared light;
attaching the fiducial target to the imaging target;
focusing a camera to image the fiducial target onto an image recording element;
reflecting the infrared light from the fiducial target to the camera;
recording a first image of the fiducial target in the image recording element;
recording a second image of the fiducial target in the image recording element;
determining a set of target motion coordinates describing six degrees of freedom of the fiducial target, based on a difference between the first image and the second image;
updating the controller with the set of target motion coordinates; and,
adjusting the magnetic resonance scanner image to correct for motion based on the set of target motion coordinates.

2. The method of claim 1 including the step of
providing a set of optical discs as the set of optical objects.

3. The method of claim 2 including the steps of:
mounting a first optical disc and a second optical disc, of the set of optical discs, on a frame in a first plane; and,
mounting a third optical disc, of the set of optical discs, on the frame in a second plane.

4. The method of claim 3 including the step of:
providing the first optical disc, the second optical disk and the third optical disk in different sizes.

5. The method of claim 3 including the step of:
arranging the second plane parallel to the first plane.

6. The method of claim 1 including the step of:
providing a set of optical rings as the set of optical objects.

7. The method of claim 1 wherein the step of attaching the fiducial target to the imaging target further comprises the steps of:
attaching the fiducial target to a means to support the fiducial target; and,
affixing the means to support the fiducial target to the imaging target.

8. The method of claim 1 wherein the step of providing a fiducial target comprises:
enabling the fiducial target as one of the group of an MRI fiducial target and an optical fiducial target.

9. The method of claim 1 including the further steps of:
providing an on-board digital image processor in the camera; and
performing the step of determining the set of target motion coordinates with the on-board digital image processor.

10. The method of claim 1 wherein the step of determining a set of target motion coordinates comprises the steps of:
determining a threshold of detected optical intensity;
identifying at least two optical objects of the set of optical objects as a first image and a second image;
calculating a set of centroid positions of each of the first image and the second image;
performing a pincushion correction to the first image and the second image;
performing a perspective correction to the first image and the second image;
extracting a set of rotation angles as a first set of target motion coordinates from the set of centroid positions;
extracting a set of translations as a second set of target motion coordinates from the set of centroid positions;
transforming the first set of target motion coordinates and the second set of target motion coordinates into a set of scanner coordinates;
converting the units of the set of scanner coordinates; and,
updating the controller with the set of scanner coordinates.

11. The method of claim 10 wherein the step of extracting a set of rotation angles images comprises the step of:
using a successive approximation means to determine rotation angles from the set of centroid positions.

12. The method of claim 1 including the steps of:
providing a projector to display a visual image on a screen which is capable of being viewed by the imaging target;
providing a two-way mirror between the imaging target and the screen; and
placing the camera adjacent the two-way mirror.

13. The method of claim 1 including the steps of:
providing an on-board digital image processor in the camera;
providing a motion monitor in communication with the on-board digital image processor;
providing a graphic user interface, in communication with the motion monitor; and,
providing an alarm function in the graphic user interface which is active when the motion monitor detects a motion condition of the imaging target.

14. The method of claim 13 wherein the step of providing an alarm function includes the step of:
providing a trend function to display a graph of the target motion parameters.

15. A system to correct motion artifacts in an MR image of a patient by a magnetic resonance scanner, the magnetic resonance scanner including a head coil surrounding the patient's head, and an MRI controller connected to the magnetic resonance scanner, the system comprising:
a fiducial target, including a set of optical objects removably secured to the patient;
a source of infrared light illuminating the fiducial target to create a reflected light pattern from the set of optical objects;
a camera comprising:
a lens positioned to receive the reflected light pattern and focus the reflected light pattern into a target image in an image plane;
an image recording element positioned in the image plane;
an on-board digital image processor electronically connected to the image recording element and to the MRI controller, the on-board digital image processor programmed to:
collect a set of target images from the image recording element,
calculate a centroid motion of the fiducial target from the set of target images, and
update the MRI controller with the centroid motion; and the MRI controller programmed to:
correct patient motion artifacts of the MR image based on the centroid motion.

16. The system of claim 15 further comprising:
a projector positioned to display a visual image on a screen which is visible by the patient; and,
a two-way mirror positioned between the patient and the screen and between the camera and the fiducial target.

17. The system of claim 15 wherein the on-board digital image processor is further programmed to:
calculate the centroid motion of the fiducial target by determining a set of target motion coordinates describing six degrees of freedom of the fiducial target based on a difference between a first image and a second image in the set of target images.

18. The system of claim 15 wherein the set of optical objects comprise circularly symmetric objects.

19. The system of claim 18 wherein the fiducial target is a set of optical discs.

20. The system of claim 19 wherein the set of optical discs comprises a first optical disc and a second optical disc mounted in a first plane; and,
a third optical disc mounted in a second plane.

21. The system of claim 20 wherein the second plane is parallel to the first plane.

22. The system of claim 20 wherein the set of optical objects includes at least one optical ring.

23. The system of claim 15 wherein the set of optical objects are of different sizes.

24. The system of claim 15 wherein the fiducial target further comprises:
a means to affix a target to the patient; and,
a target means for targeting one of the group of light and MRI.

25. The system of claim 24 wherein a stalk supports a water containing the fiducial target.

26. The system of claim 17 wherein the on-board digital image processor is programmed to:
determine a threshold of detected optical intensity;
identify at least two of the set of optical objects as imaged objects;
calculate a set of centroid positions of each of the imaged objects;
perform a pincushion correction to the first image and the second image;
perform a perspective correction to the first image and the second image;
extract a set of rotation angles from the set of centroid positions;
extract a set of translations from the set of centroid positions;
transform the set of rotation angles and the set of translations to a set of scanner coordinates;
convert the units of the set of scanner coordinates; and,
update the MRI controller with the set of scanner coordinates.

27. The system of claim 26 wherein the on-board digital image processor is further programmed with a successive approximation means to extract the set of rotation angles.

28. The system of claim 15 further comprising a graphical user interface included in a motion monitor programmed to effect an alarm of excessive patient motion based on the centroid motion.

29. The system of claim 28 wherein the graphical user interface includes an alarm function.

30. The system of claim 28 wherein the graphical user interface is further programmed with a trend function to display a graph of the centroid motion.

31. A method for patient motion correction in a magnetic resonance scanner, the magnetic resonance scanner including a head coil adjacent a patient's head during creation of an MRI scan image, and a controller, the method comprising the steps of:
providing a source of infrared light;
providing a fiducial target comprising a set of optical objects;
attaching the fiducial target to the patient;
focusing a camera with an on-board digital image processor on the fiducial target;
reflecting the infrared light from the fiducial target to the camera;
recording a first image of the fiducial target in the camera;
recording a second image of the fiducial target in the camera;
selectively enabling a tracking function;
collecting a time series of images from the camera;
tracking a centroid position for the time series of images, characterized by six degrees of freedom of the fiducial target;
updating the controller with the centroid position; and,
adjusting the MRI scan image based on the centroid position.

32. A method for motion detection of a patient while in a magnetic resonance scanner, the magnetic resonance scanner including a head coil adjacent to a patient's head during creation of an MRI scan image, and a controller, the method comprising the steps of:
providing a source of infrared light;
providing a fiducial target comprising a set of optical objects;
attaching the fiducial target to the patient;
focusing a camera on the fiducial target;
providing a motion monitor in communication with an on-board digital image processor;
programming a graphical user interface with a trend selector and an alarm selector;
reflecting the infrared light from the fiducial target to the camera;
recording a first image of the fiducial target in the camera;
recording a second image of the fiducial target in the camera;
setting a rotational threshold;
setting a translational threshold;
collecting a time series of images;
tracking a centroid motion, characterized by six degrees of freedom, for the fiducial target;
updating the motion monitor with the centroid motion;
displaying a graph of centroid motion on a display screen;
comparing the centroid motion to the rotational threshold and to the translational threshold; and,
indicating an alarm if the centroid motion exceeds at least one of the rotational threshold and the translational threshold.
